(19) United States
(12) Patent Application Publication
(10) Pub. No.: US 2026/0014389 A1
(43) Pub. Date: Jan. 15, 2026

(54) DEVICE FOR TREATING SKIN BY USING LASER BEAMS OF MULTIPLE WAVELENGTHS AND OPERATION METHOD THEREOF

(71) Applicant: HEALUX CO.,LTD., Seoul (KR)

(72) Inventor: Do Yeun KIM, Seoul (KR)

(73) Assignee: HEALUX CO.,LTD., Seoul (KR)

(21) Appl. No.: 19/332,160

(22) Filed: Sep. 18, 2025

Related U.S. Application Data
(63) Continuation of application No. PCT/KR2023/003677, filed on Mar. 20, 2023.

(51) Int. Cl. A61N 5/06; A61N 5/067

(52) U.S. Cl. CPC A61N 5/0617; A61N 5/067; A61N 2005/0626; A61N 2005/0644; A61N 2005/0651; A61N 2005/066

(57) ABSTRACT

Provided is a medical skin treatment device including a control unit for controlling the operation of the skin treatment device; a main body which includes a transfer unit and a power supply unit; and a handpiece which is supplied with power from the power supply unit and controlled by the control unit and one side of which is brought into contact with a patient's skin to irradiate same with laser beams. The handpiece includes at least four different laser diodes; the at least four laser diodes radiate laser beams of four different wavelengths and comprise first laser diodes, second laser diodes, third laser diodes, and fourth laser diodes; the number of the first laser diodes is 30 percentage or less of the total number of the laser diodes; and the number of the fourth laser diodes is 25 percentage or greater of the total number of the laser diodes.

### CROSS-REFERENCE TO RELATED APPLICATION

This application is a continuation of International Application No. PCT/KR2023/003677 filed on Mar. 20, 2023, the entire contents of which are herein incorporated by reference.

### TECHNICAL FIELD

The present disclosure relates to a skin treatment device that is automatically controlled according to skin conditions. The present disclosure also relates to a skin treatment device with an improved transducer and a skin treatment device using electrodes. The present disclosure also relates to a skin treatment device for medication injection.

### BACKGROUND ART

Recently, interest in skin treatments has been growing in popularity. Accordingly, various type of skin treatment devices have been developed to improve skin elasticity, reduce wrinkles, and improve the overall appearance of the skin. One of the most popular skin treatments is a laser treatment. The laser treatment is a noninvasive treatment that can be used to restore skin elasticity.

There are many different types of laser beams available for skin treatments, each with its own unique characteristics. The effectiveness of laser treatments may vary depending on several factors, such as a wavelength of laser beams, an exposure time, and energy used. However, since conventional skin treatment devices are designed for specific purposes, it has been very difficult for hospitals to equip the devices according to their purpose. Therefore, a medical skin treatment device that can restore the elasticity of a patient's skin, is easy to use, and can provide effective and versatile laser treatments to the patient is required.

### DISCLOSURE

### Technical Problem

The present disclosure relates to a medical skin treatment device capable of effectively treating various types of patients' skin and an easy-to-use medical skin treatment device. However, objects according to the technical spirit of the present disclosure are not limited to the above-described object and other objects may be present.

### Technical Solution

A medical skin treatment device according to the present disclosure includes a control unit configured to control the operation of the skin treatment device, a main body which includes a transfer unit and a power supply, and a handpiece which receives power from the power supply of the main body and is controlled by the control unit and of which one side is brought into contact with a patient's skin to irradiate the skin with laser beams, wherein the handpiece includes at least four different laser diodes, the at least four laser diodes radiate laser beams of four different wavelengths and include first laser diodes, second laser diodes, third laser diodes, and fourth laser diodes, the number of the first laser diodes is 30 percent or less of the total number of the laser diodes, and the number of the fourth laser diodes is 25 percent or greater of the total number of the laser diodes.

In the medical skin treatment device according to the present disclosure, the first laser diodes may be laser diodes for burning hair on a surface of the patient's skin, the second to fourth laser diodes may be laser diodes for penetrating the patient's skin to form a lesion under the patient's skin, a depth at which the second laser diode penetrates into the skin may be smaller than a depth at which the third laser diode penetrates into the skin, and the depth at which the third laser diode penetrates into the skin may be smaller than a depth at which the fourth laser diode penetrates into the skin.

In the medical skin treatment device according to the present disclosure, a wavelength of light generated from the first laser diode may be the shortest, a wavelength of light generated from the second laser diode may be longer than the wavelength of the light generated from the first laser diode, a wavelength of light generated from the third laser diode may be longer than the wavelength of the light generated from the second laser diode, and a wavelength of light generated from the fourth laser diode may be longer than the wavelength of the light generated from the third laser diode.

In the medical skin treatment device according to the present disclosure, the wavelength of the light generated from the first laser diode may be 750 nanometers or more and 770 nanometers or less, the wavelength of the light generated from the second laser diode may be 800 nanometers or more and 820 nanometers or less, the wavelength of the light generated from the third laser diode may be 930 nanometers or more and 950 nanometers or less, and the wavelength of the light generated from the fourth laser diode may be 1,050 nanometers or more and 1,070 nanometers or less.

In the medical skin treatment device according to the present disclosure, the first laser diodes may be included in a first laser module, the second laser diodes may be included in a second laser module, the third laser diodes may be included in a third laser module, the fourth laser diodes may be included in a fourth laser module, and the first to fourth laser modules may be arranged to be coplanar with the handpiece.

In the medical skin treatment device according to the present disclosure, the first laser module, the second laser module, the third laser module, and the fourth laser module may have a form of laser diodes arranged on a rectangular substrate, wherein the rectangular substrate has long sides in a first direction and short sides in a second direction perpendicular to the first direction, the first laser module, the second laser module, the third laser module, and the fourth laser module may be arranged in parallel in the second direction to form a laser module array, and the laser module array may include five first laser modules, four second laser modules, five third laser modules, and six fourth laser modules.

In the medical skin treatment device according to the present disclosure, the control unit may control an intensity of light generated from the first laser module to be greater than an intensity of light generated from any one of the second to fourth laser modules for a predetermined first period of time, and operate the first to fourth laser modules according to a predetermined pattern after the first period of time.

In the medical skin treatment device according to the present disclosure, the handpiece may include a skin imaging camera that is positioned to be coplanar with the first laser module, the second laser module, the third laser module, and the fourth laser module and photographs the patient's skin, when the skin imaging camera photographs a start point marker pre-marked on the surface of the patient's skin, the control unit may control any one of the first to fourth laser modules to be turned on, and when the skin imaging camera photographs an end point marker pre-marked on the surface of the patient's skin, the control unit may control any one of the first to fourth laser modules to be turned off.

Further, a program for implementing an operating method of the skin treatment device as described above may be recorded on a computer-readable recording medium.

### Advantageous Effects

A skin treatment device of the present disclosure can effectively treat various skin conditions in patients. Therefore, there is no need to provide a plurality of skin treatment devices. Further, when there are many functions, the settings can become complicated, but the skin treatment device of the present disclosure can semi-automatically set the settings according to the conditions of the patient's skin, thereby increasing convenience for a practitioner.

Effects obtainable in the present disclosure are not limited to the above-described effects and other effects that are not described may be clearly understood by those skilled in the art from the following descriptions.

### DESCRIPTION OF DRAWINGS

FIG. 1 is a diagram illustrating a medical skin treatment device according to an embodiment of the present disclosure.

FIG. 2 is a block diagram illustrating various components that may be included in a skin treatment device according to an embodiment of the present disclosure.

FIG. 3 is a diagram for describing a handpiece of a skin treatment device according to an embodiment of the present disclosure.

FIG. 4 is a diagram for describing a laser module according to an embodiment of the present disclosure.

FIG. 5 is a diagram for describing a handpiece according to an embodiment of the present disclosure.

FIGS. 6A and 6B show diagrams for describing a handpiece according to an embodiment of the present disclosure.

FIG. 7 illustrates an operating method of a skin treatment device according to an embodiment of the present disclosure.

FIG. 8 is a diagram for describing the operation of a skin treatment device according to an embodiment of the present disclosure.

FIG. 9 is a diagram for describing a handpiece according to an embodiment of the present disclosure.

FIG. 10 is a diagram for describing markers according to an embodiment of the present disclosure.

FIG. 11 is a diagram illustrating an example of performing a laser treatment using markers according to an embodiment of the present disclosure.

FIGS. 12A and 12B show diagrams for describing a handpiece according to an embodiment of the present disclosure.

### MODES OF THE INVENTION

Advantages and features of disclosed embodiments and methods of achieving the same will be clearly understood with reference to the accompanying drawings and embodiments described in detail below. However, the present disclosure is not limited to the embodiments to be disclosed below and may be implemented in various different forms. The embodiments are provided in order to fully explain the present embodiments and fully explain the scope of the present disclosure for those skilled in the art.

Terms used in this specification will be briefly described, and the disclosed embodiments will be described in detail.

Although the terms used herein are selected from among general terms that are currently and widely used in consideration of functions in embodiments of the present disclosure, these may be changed according to the intentions of those skilled in the art, precedents, or the advent of new technology. In addition, in a specific case, some terms may be arbitrarily selected by applicants. In this case, meanings thereof will be described in detail in a corresponding description of embodiments of the present disclosure. Therefore, the terms used herein should be defined based on the meanings of the terms and content of this entire specification, rather than simply the terms themselves.

As used herein, the singular forms “a” and “an” are intended to also include the plural forms, unless the context clearly indicates otherwise. Further, the plural forms are intended to also include the singular forms, unless the context clearly indicates otherwise.

Throughout this specification, when a part “includes” an element, another element may be further included, rather than excluding the presence of the other element, unless otherwise described.

Further, the term “unit” described in the specification refers to software or a hardware component, and the unit performs certain functions. However, the “unit” is not limited to software or hardware. The “unit” may be configured in a storage medium that may be addressed or may be configured to be executed by at least one processor. Therefore, examples of the “unit” include components such as software components, object-oriented software components, class components and task components, and processes, functions, attributes, procedures, subroutines, segments of program codes, drivers, firmware, micro codes, circuits, data, databases, data structures, tables, arrays, and variables. Components and functions provided from “units” may be combined into a smaller number of components and “units” or may be further separated into additional components and “units.”

According to an embodiment of the present disclosure, “unit” may be implemented with a processor or a memory. The term “processor” should be interpreted broadly to include a general purpose processor, a central processing unit (CPU), a microprocessor, a digital signal processor (DSP), a controller, a microcontroller, a state machine, or the like. In some environments, “processor” may refer to an application-specific integrated circuit (ASIC), a programmable logic device (PLD), a field-programmable gate array (FPGA), or the like. The term “processor” may refer to, for example, combinations of processing devices, such as a combination of a DSP and a microprocessor, a combination of a plurality of microprocessors, a combination of one or more microprocessors in combination with a DSP core, and a combination of any other such components.

The term “memory” should be interpreted broadly to include any electronic component that can store electronic information. The term “memory” may refer to any of various types of processor-readable media, such as a random-access memory (RAM), a read-only memory (ROM), a non-volatile random-access memory (NVRAM), a programmable read-only memory (PROM), an erasable PROM (EPROM), an electrically erasable PROM (EEPROM), a flash memory, a magnetic or optical data storage, a register, and the like. When a processor is capable of reading information from and/or writing information to a memory, the memory is determined to be in electronic communication with the processor. A memory integrated in a processor is in electronic communication with the processor.

In this specification, an actuator is a component capable of providing driving force. For example, the actuator may include a motor, a linear motor, an electric motor, a DC motor, an AC motor, a linear actuator, an electric actuator, and the like, but the present disclosure is not limited thereto.

In the present disclosure, the term “downward” may refer to a direction from a skin treatment device 100 toward the ground, and the term “upward” may refer to a direction from the ground toward the skin treatment device 100.

Hereinafter, embodiments of the present disclosure that may be easily performed by those skilled in the art will be described in detail with reference to the accompanying drawings. In addition, in the drawings, parts irrelevant to the description will be omitted to clearly describe the present disclosure.

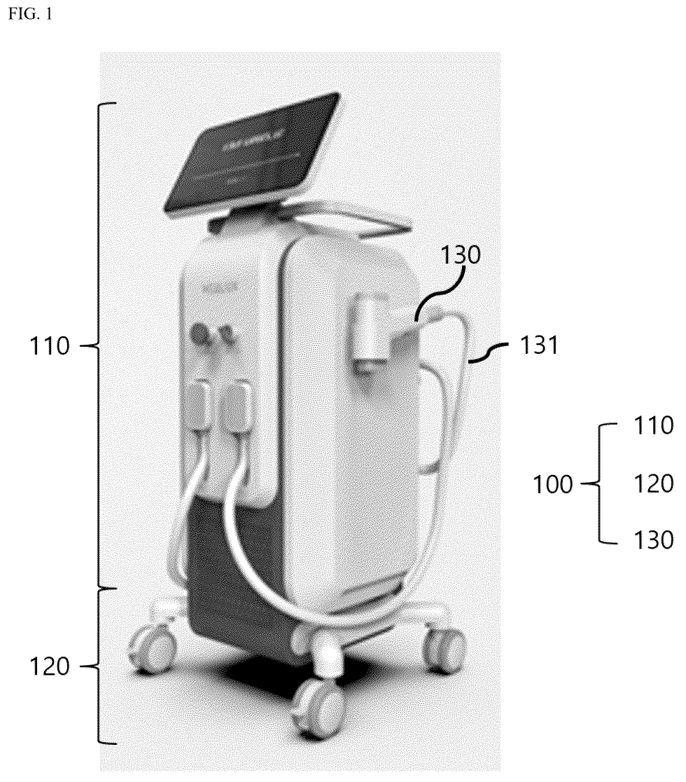

FIG. 1 is a diagram illustrating a medical skin treatment device according to an embodiment of the present disclosure.

A medical skin treatment device 100 may include a control unit 200 and a main body 110 including a power supply. The main body 110 may further include at least one of a communication unit, a sensor unit, an output unit, and an input unit.

Further, the medical skin treatment device 100 may include a transfer unit 120 for moving the main body 110. The transfer unit 120 may be positioned on a lower end of the main body 110. The transfer unit 120 may include at least one wheel. The transfer unit 120 may include at least one of a caster wheel, an electric wheel, and a Mecanum wheel. The transfer unit 120 may include an electric motor, and the electric motor may provide driving force on the basis of a user input. Further, the transfer unit 120 may be autonomously moved under the control of the control unit 200. However, the present disclosure is not limited thereto, and the transfer unit 120 may be moved by the user's power.

The power supply may be a component for supplying power to the medical skin treatment device 100. The power supply may include a battery. The power supply may include a component for converting a voltage of the battery into a voltage suitable for the skin treatment device 100. Further, according to various embodiments of the present disclosure, the power supply may be a device for converting power supplied from an external device into a voltage suitable for the skin treatment device 100, without including a battery.

Further, the medical skin treatment device 100 may include a handpiece 130. The main body 110 may be connected to the handpiece 130 through a cable 131. The handpiece 130 may be used to treat a patient's skin. The handpiece 130 may include a handle that the user can hold. One side of the handpiece 130 may have a surface that is formed to be brought into contact with the patient's skin. The main body 110 may supply power to the handpiece 130 through the cable 131 and transmit or receive control signals in a wired manner. The handpiece 130 may receive power from the power supply of the main body 110 and may be controlled by the control unit 200. The one side of the handpiece 130 may be brought into contact with the patient's skin to irradiate the skin with laser beams. The one side of the handpiece 130 may be a surface equipped with a laser diode for radiating laser beams.

Hereinafter, each function of the skin treatment device 100 will be described.

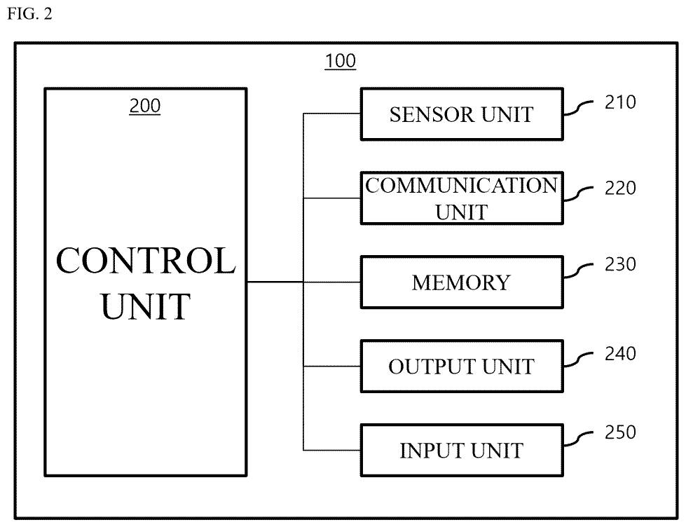

FIG. 2 is a block diagram illustrating various components that may be included in a skin treatment device according to an embodiment of the present disclosure.

A skin treatment device 100 may include a sensor unit 210, a communication unit 220, a memory 230, an output unit 240, an input unit 250, and a control unit 200.

The skin treatment device 100 may include the control unit 200. The control unit 200 may control the operation of the skin treatment device 100. For example, the skin treatment device 100 may include a control unit 200 for controlling the operation of at least one of a wheel that can drive a main body 110 and a handpiece. The control unit 200 may include one processor or a plurality of processors. The control unit 200 may be included in the main body 110. When the control unit 200 includes a plurality of processors, at least some of the plurality of processors may be physically provided at positions spaced away from the main body 110. Further, the skin treatment device 100 is not limited thereto and may be implemented by various ways.

According to an embodiment of the present disclosure, the control unit 200 may control the operation of the skin treatment device 100. For example, the skin treatment device 100 may include a plurality of actuators, and the skin treatment device 100 may control the operation of the skin treatment device 100 by controlling the operation of the plurality of actuators. For example, the control unit 200 may control the operation of the handpiece 130 to perform an operation for improving the patient's skin.

The skin treatment device 100 may include the sensor unit 210. The sensor unit 210 may acquire various information using at least one sensor. The sensor unit 210 may be equipped with a sensor that uses a measurement method such as pressure, electrical potential, and optics, etc. For example, the sensor unit 210 may include at least one of a distance measurement sensor, a movement distance measurement sensor, and an encoder. Further, the sensor unit 210 may include a pressure sensor, an infrared sensor, a light-emitting diode (LED) sensor, a touch sensor, etc. However, the present disclosure is not limited thereto. The sensor unit 210 may be included in at least one of the main body 110, the transfer unit 120, and the handpiece 130.

Further, the skin treatment device 100 may include the communication unit 220. The communication unit 220 may be a component for enabling the skin treatment device 100 to communicate with internal modules or external devices in a wired or wireless manner. The external devices may include external servers or user terminals. The user terminals may include personal computers (PCs), smartphones, tablet computers, or wearable devices. The communication unit 220 may include wired/wireless communication modules for network access. Wireless communication technologies may include, for example, a wireless local area network (WLAN), Wi-Fi, wireless broadband (WiBro), World Interoperability for Microwave Access (WiMAX), High Speed Downlink Packet Access (HSDPA), etc. Wired communication technologies may include, for example, a digital subscriber line (XDSL), fiber-to-the-home (FTTH), power line communication (PLC), etc. Further, a network connection unit may include a short-range communication module and transmit or receive data to or from any device/terminal positioned in close proximity. Short-range communication technologies may include, for example, Bluetooth, radio frequency identification (RFID), Infrared Data Association (IrDA), ultra-wideband (UWB), Zigbee, etc., but the present disclosure is not limited thereto.

The skin treatment device 100 may include the memory 230. The control unit 200 may execute commands stored in the memory 230. The memory 230 may be included in the control unit 200 or provided outside the control unit 200. The memory 230 may store various information related to the skin treatment device 100. For example, the memory 230 may include information related to an operating method of the handpiece 130, captured images, and user authentication information, but the present disclosure is not limited thereto.

The memory 230 may be implemented using a non-volatile storage medium capable of persistently storing arbitrary data. For example, the memory 230 may include a storage device based on a flash memory and/or a battery-backed memory as well as a disk, an optical disc, and a magneto-optical storage device, but the present disclosure is not limited thereto. The memory 230 may be a primary storage device that a processor directly accesses, such as a random access memory (RAM) such as a dynamic random access memory (DRAM), a static random access memory (SRAM), etc., and may be a volatile storage device in which stored information is instantly erased when power is turned off, but the present disclosure is not limited thereto. The memory 230 may be operated by the control unit 200.

Further, the skin treatment device 100 may further include a manipulation unit that provides an interface for manipulating the skin treatment device 100. The manipulation unit may include the output unit 240 and the input unit 250.

The output unit 240 may output information related to treatments, information related to patients, information related to the control of the handpiece, and information related to the state of the main body in the form of sound and images under the control of the control unit 200. The output unit 240 may include a speaker or a display. The output unit 240 may output acquired medical images. The output unit 240 may output information necessary for the user to manipulate the skin treatment device 100, such as a user interface (UI), user information, object information, etc. Examples of the output unit 240 may include a speaker, a printer, a cathode-ray tube (CRT) display, a liquid-crystal display (LCD) display, a plasma display panel (PDP) display, an organic light-emitting diode (OLED) display, a field-emission display (FED) display, an LED display, a vacuum fluorescent display (VFD) display, a digital light processing (DLP) display, a flat-panel display (FPD), a three-dimensional (3D) display, a transparent display, etc., and may include various output devices within a range obvious to those skilled in the art.

The output unit 240 may display information related to treatment. The information related to treatment may include at least one of a treatment method to be provided to the patient, a treatment time period, and information related to the patient's disease. The treatment method indicates the energy used to treat the skin and may include a laser treatment. The output unit 240 may include information related to the patient. The information related to the patient may include at least one of the patient's age, gender, treatment period, and disease-related information. Information related to the control of the handpiece may include at least one of a button for controlling the handpiece, an operating cycle, the type of base unit currently connected to the handpiece, the intensity of laser beams used for treatment, and the frequency of a laser radiation pulse. Information related to the state of the main body may include at least one of the state of the battery of the main body, a power connection state, and an error state.

Further, the output unit 240 may output information related to the control of the handpiece 130. For example, when the handpiece 130 is in operation, the control unit 200 may control the output unit 240 to output sound or light so that people in the vicinity can know that the handpiece is in operation.

The skin treatment device 100 may be connected to a workstation in a wired or wireless manner. The workstation may be present in a space physically separated from the skin treatment device 100.

The workstation may include a storage server. The storage server may store at least one of information about the patient, information about the treatment, and information about the user (medical professional). The workstation may include a review device. The review device may receive medical images from the storage server and display the medical images on the basis of user commands. The workstation and the skin treatment device 100 may transmit, store, process, and output data according to the Digital Imaging and Communications in Medicine (DICOM) standard. Further, the workstation may include a picture archiving and communication system (PACS).

The workstation may include an output unit, an input unit, and a control unit. The output unit and the input unit provide an interface for manipulating the workstation and the skin treatment device 100 to the user. The control unit of the workstation may control the workstation and the skin treatment device 100.

The skin treatment device 100 may be controlled through the workstation or by the control unit 200 included in the skin treatment device 100. Therefore, the user may control the skin treatment device 100 through the workstation or control the skin treatment device 100 through the manipulation unit and control unit 200 that are included in the skin treatment device 100. In other words, the user may remotely control the skin treatment device 100 through the workstation or directly control the skin treatment device 100.

The control unit of the workstation and the control unit 200 of the skin treatment device 100 may be separate, but the present disclosure is not limited thereto. The control unit of the workstation and the control unit 200 of the skin treatment device 100 may be implemented as a single integrated control unit, and the integrated control unit may be included in only one of the workstation and the skin treatment device 100. Hereinafter, the control unit 200 may be the control unit of the workstation and/or the control unit of the skin treatment device 100.

The output unit and input unit of the workstation and the output unit 240 and input unit 250 of the skin treatment device 100 may each provide an interface for manipulating the skin treatment device 100 to the user. The workstation and the skin treatment device 100 may each include the output unit and the input unit, but the present disclosure is not limited thereto. The output unit or the input unit may be implemented in only one of the workstation and the skin treatment device 100.

Hereinafter, the input unit 250 is the input unit of the workstation and/or the input unit of the skin treatment device 100, and the output unit 240 is the output unit of the workstation and/or the output unit of the skin treatment device 100.

The input unit 250 may receive commands for manipulating the skin treatment device 100 and various types of information related to X-ray photography from the user. The control unit 200 may control or manipulate the skin treatment device 100 on the basis of the information input into the input unit 250. The input unit 250 may include a joystick, a keyboard, a mouse, a touch screen, a shutter button, an unlock button, a voice recognition device, a fingerprint recognition device, an iris recognition device, etc., and include other input devices obvious to those skilled in the art. The user may input commands for operating the handpiece through the input unit 250, and a switch for inputting such commands may be provided in the input unit 250.

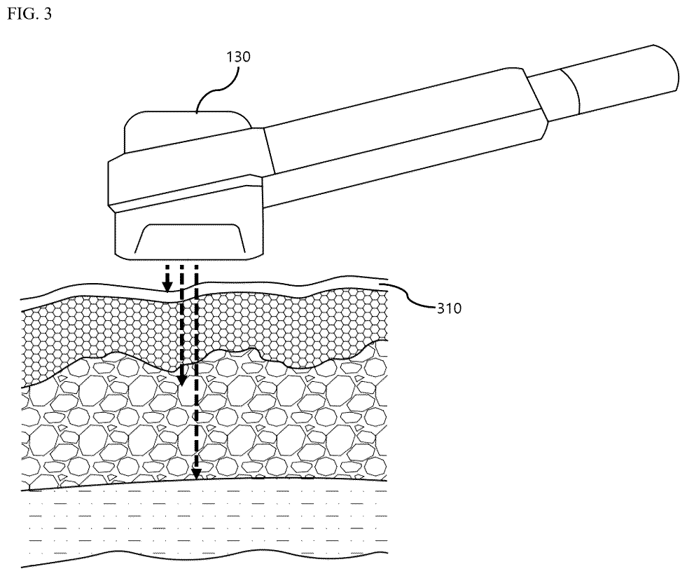

FIG. 3 is a diagram for describing a handpiece of a skin treatment device according to an embodiment of the present disclosure.

One side of a handpiece 130 may be brought into contact with a patient's skin 310 to irradiate the skin 310 with laser beams. The handpiece 130 may include at least four different laser diodes. The control unit 200 may supply power received from the power supply to the at least four laser diodes. The control unit 200 may supply power to at least one of the at least four laser diodes to turn on at least one of the at least four laser diodes. The skin treatment device 100 may treat the patient's skin by turning on or off the at least four laser diodes according to a predetermined pattern.

The at least four laser diodes may radiate laser beams of four different wavelengths. For example, the at least four laser diodes may include a first laser diode, a second laser diode, a third laser diode, and a fourth laser diode. The first laser diode, the second laser diode, the third laser diode, and the fourth laser diode may all be components for treating the skin. For example, the first laser diode, the second laser diode, the third laser diode, and the fourth laser diode may be components for improving skin elasticity and removing wrinkles.

However, the first laser diode, the second laser diode, the third laser diode, and the fourth laser diode may perform different functions to improve the patient's skin. For example, the first laser diode may be a laser diode for burning hair on a surface of the patient's skin 310. The handpiece 130 may remove the hair on the surface of the patient's skin 310 using the first laser diode and thus allow light from the second to fourth laser diodes to effectively reach an interior of the patient's skin 310. Therefore, the skin improvement effect may be further increased by the second to fourth laser diodes.

The first to fourth laser diodes included in the skin treatment device 100 may use energy of laser beams to heat collagen fibers in the skin, thereby contracting and tightening the patient's skin 310. For example, the energy of the laser beams may form lesions in collagen fibers, and as these lesions are healed, skin elasticity can be improved. This is because the process of healing the lesions formed in the patient's skin 310 by the laser beams stimulates the generation of new collagen fibers.

The first to fourth laser diodes of the handpiece 130 may emit light of a specific wavelength, which is absorbed by skin moisture and chromophores, to heat collagen fibers. Laser diodes typically use fractional laser technology, which means that energy of laser beams is transmitted to the patient's skin 310 in a pattern of small dots or columns. This allows for targeted treatment of a specific area of the patient's skin 310 without affecting surrounding tissues where the laser beams do not reach. Further, fractional technology may achieve faster healing and minimize downtime compared to conventional laser treatments. However, the present disclosure is not limited thereto, and various laser technologies may be used.

Laser beams of the second to fourth laser diodes may penetrate into the patient's skin to form lesions beneath the patient's skin. The lesions may have cylindrical shapes. The lesions may be formed by damaging the patient's skin 310 with the energy of the second to fourth laser diodes. The lesions may be formed beneath the patient's skin 310. The patient's skin 310 may have wrinkles removed and skin elasticity improved by treating the lesions.

The lasers beam of the second to fourth laser diodes may penetrate into the patient's skin 310 at different depths. For example, the depth at which the laser beam of the second laser diode penetrates into the skin may be smaller than the depth at which the laser beam of the third laser diode penetrates into the skin. Further, the depth at which the laser beam of the third laser diode penetrates into the skin may be smaller than the depth at which the laser beam of the fourth laser diode penetrates into the skin. In this way, by including the laser diodes of which the laser beams penetrate into the skin at different depths, the handpiece 130 may effectively form lesions from shallow to deep skin areas. Therefore, the skin improvement effect may be maximized.

In order to maximize the skin improvement effect, the number of laser diodes may vary depending on the type thereof. For example, the number of first laser diodes may be 10 percent or greater and 30 percent or less of the total number of laser diodes. The total number of laser diodes may be the number of all laser diodes included in first to fourth laser modules. The total number of laser diodes may be the number of all laser diodes included in the handpiece 130. Further, the number of fourth laser diodes may be 25 percent or greater and 40 percent or less of the total number of laser diodes. What is important here is that the number of first laser diodes is 30 percent or less of the total number of laser diodes, and the number of fourth laser diodes is 25 percent or greater of the total number of laser diodes. The skin treatment device 100 of the present disclosure includes the laser diodes in the above ratios, thereby maximizing the improvement effect on the patient's skin 310, and this has been experimentally proven.

Although the ratio of the number of first laser diodes to the number of fourth laser diodes has been described above, the present disclosure is not limited thereto. Instead of the ratio of the number of first laser diodes to the number of fourth laser diodes, a ratio of an area or light quantity of the first laser diodes to an area or light quantity of the fourth laser diodes may be used. For example, the area or light quantity of the first laser diodes may be 10 percent or greater and 30 percent or less of the total area or light quantity of the laser diodes. Further, the area or light quantity of the fourth laser diodes may be 25 percent or greater and 40 percent or less of the total area or light quantity of the laser diodes.

The number of second laser diodes may be 15 percent or greater and 25 percent or less of the total number of laser diodes. Further, the number of third laser diodes may be 25 percent or greater and 35 percent or less of the total number of laser diodes. The number of second laser diodes may be smaller than the number of third laser diodes. Since the laser beams have continuous energy, continuous lesions may be formed from the surface of the patient's skin 310 to the deepest area the laser beams may reach. When the patient's skin is thin, at least one of the second laser diode and the third laser diode may be used instead of the fourth laser diode. That is, the fourth laser diode may be deactivated and at least one of the second laser diode and the third laser diode may be activated.

However, the present disclosure is not limited thereto, and at least two of the second to fourth laser diodes may be used together. The lesions formed on the surface of the patient's skin 310 may harm the patient's skin. Therefore, the skin treatment device 100 may include a handpiece 130 including a cooling unit 1210 to prevent damage to the surface of the patient's skin 310. Since the surface of the patient's skin 310 is cooled by the cooling unit 1210, the closer the device is to the surface, the less effective the skin improvement effect by the laser beams may be. Therefore, the handpiece 130 may apply more energy to a shallow area of the patient's skin 310 using at least two of the second to fourth laser diodes. The handpiece 130 according to the present disclosure may include second laser diodes in an amount of 15 percent or greater and 25 percent or less of the total number of laser diodes and include third laser diodes in an amount of 25 percent or greater and 35 percent or less of the total number of laser diodes. According to the ratios of the second and third laser diodes as described above, the interference with the skin improvement effect caused by the cooling unit 1210 may be overcome. Therefore, the skin treatment device 100 of the present disclosure may maximize the improvement effect on the patient's skin 310.

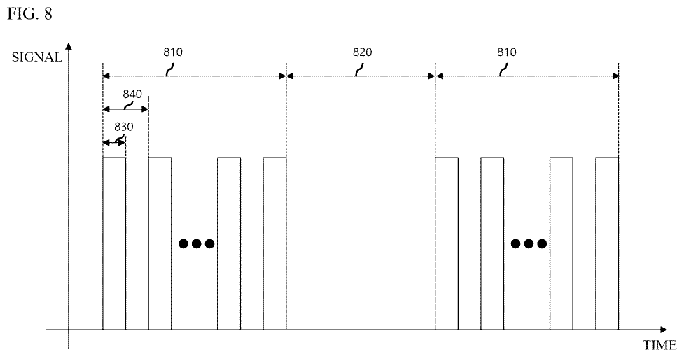

Although the ratio of the number of first laser diodes to the number of fourth laser diodes has been described above, the present disclosure is not limited thereto. Instead of the ratio of the number of first laser diodes to the number of fourth laser diodes, at least one of the ratio of the areas, the ratio of light quantity, and the ratio of radiation times of the first to four laser diodes may be used. For example, at least one of the ratio of the area, the light quantity, and the radiation time of the first laser diode may be 10 percent or greater and 30 percent or less of at least one of the total area, light quantity, and radiation time of the laser diodes. At least one of the ratio of the area, the light quantity, and the radiation time of the second laser diodes may be 15 percent or greater and 25 percent or less of at least one of the total area, light quantity, and radiation time of the laser diodes. At least one of the ratio of the area, the light quantity, and the radiation time of the third laser diodes may be 25 percent or greater and 35 percent or less of at least one of the total area, light quantity, and radiation time of the laser diodes. Further, the area or light quantity of the fourth laser diode may be 25 percent or greater and 40 percent or less of the total area or light quantity of the laser diodes. A radiation time period 810 will be described with reference to FIG. 8. The radiation time period 810 may be replaced by a pulse-on time period 830 divided by a pulse cycle 840. That is, instead of the ratio of the number of first laser diodes to the number of fourth laser diodes, the pulse-on time period 830 of the first to fourth laser diodes divided by the pulse cycle 840 may be used.

According to an embodiment of the present disclosure, a wavelength of light generated from the first laser diode may be the shortest. For example, the wavelength of the light generated from the first laser diode may be 750 nanometers or greater and 770 nanometers or less. More specifically, the wavelength of the light generated from the first laser diode may be about 760 nanometers.

Further, a wavelength of light generated from the second laser diode may be longer than the wavelength of the light generated from the first laser diode. The wavelength of the light generated from the second laser diode may be shorter than a wavelength of light generated from the third laser diode. For example, the wavelength of the light generated from the second laser diode may be 800 nanometers or greater and 820 nanometers or less. More specifically, the wavelength of the light generated from the second laser diode may be about 810 nanometers.

Further, the wavelength of the light generated from the third laser diode may be longer than the wavelength of the light generated from the second laser diode. The wavelength of the light generated from the third laser diode may be shorter than a wavelength of light generated from the fourth laser diode. For example, the wavelength of the light generated from the third laser diode may be 930 nanometers or greater and 950 nanometers or less. More specifically, the wavelength of the light generated from the third laser diode may be about 940 nanometers.

Further, the wavelength of the light generated from the fourth laser diode may be longer than the wavelength of the light generated from the third laser diode. For example, the wavelength of the light generated from the fourth laser diode may be 1,050 nanometers or greater and 1,070 nanometers or less. More specifically, the wavelength of the light generated from the fourth laser diode may be about 1,064 nanometers.

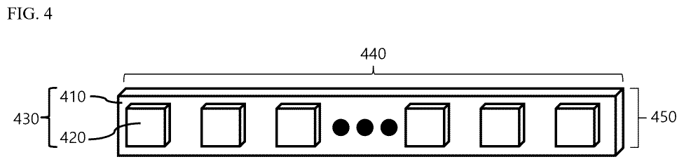

The laser diodes may be mounted on a laser module. The laser module will be described with reference to FIG. 4.

FIG. 4 is a diagram for describing a laser module according to an embodiment of the present disclosure.

One or more laser diodes 420 may be mounted on a substrate 410 to form a laser module 430. The same type of laser diodes 420 may be mounted on a single substrate 410. That is, the laser module 430 may include a laser diode that outputs laser beams of a single wavelength. However, the present disclosure is not limited thereto, and various types of laser diodes 420 may be mounted on the single substrate 410. Further, the laser module 430 may output laser beams of various wavelengths.

The laser diodes 420 may include a first laser diode, a second laser diode, a third laser diode, and a fourth laser diode. Further, the laser module 430 may include a first laser module, a second laser module, a third laser module, and a fourth laser module.

The first laser diode may be included in the first laser module. The second laser diode may be included in the second laser module. The third laser diode may be included in the third laser module. The fourth laser diode may be included in the fourth laser module. Therefore, the first laser module may emit light having the same wavelength as the first laser diode, the second laser module may emit light having the same wavelength as the second laser diode, the third laser module may emit light having the same wavelength as the third laser diode, and the fourth laser module may emit light having the same wavelength as the fourth laser diode.

However, the present disclosure is not limited thereto, and at least one of the first to fourth laser diodes may be included in the first laser module, at least one of the first to fourth laser diodes may be included in the second laser module, at least one of the first to fourth laser diodes may be included in the third laser module, and at least one of the first to fourth laser diodes may be included in the fourth laser module.

Referring to FIG. 4, the laser diodes 420 are arranged in parallel in a line on the substrate 410. However, the present disclosure is not limited thereto, and the laser diodes 420 may be formed in a plurality of rows on the substrate 410.

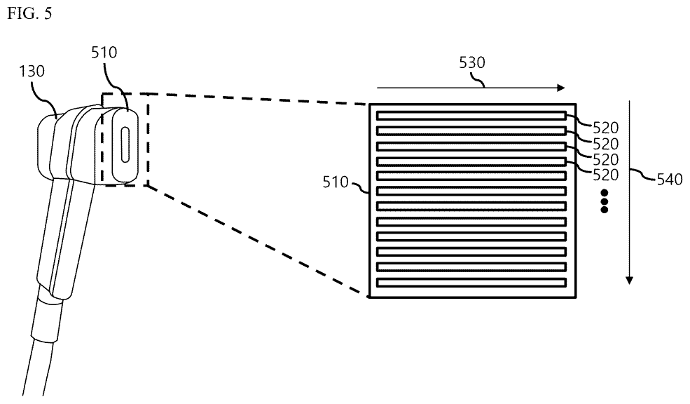

The first to fourth laser modules may be arranged to be coplanar with a handpiece. This will be described with reference to FIG. 5.

FIG. 5 is a diagram for describing a handpiece according to an embodiment of the present disclosure.

Referring to FIG. 5, a surface 510 on one side of a handpiece 130 may include a plurality of laser modules. The plurality of laser modules may include at least one of a first laser module, a second laser module, a third laser module, and a fourth laser module.

Referring briefly to FIG. 4, the first laser module, the second laser module, the third laser module, and the fourth laser module may be formed such that laser diodes 420 are arranged on a rectangular substrate 410. The rectangular substrate 410 may have a long side 440 in a first direction 530 and a short side 450 in a second direction 540 perpendicular to the first direction. In this way, the long side 440 may have a length that is at least five times or more the length of the short side 450.

Referring to FIG. 5, the first laser module, the second laser module, the third laser module, and the fourth laser module may be arranged in parallel in the second direction 540 to form a laser module array. As illustrated in FIG. 5, a long side 440 may have a length that is at least five times or more the length of a short side 450, and laser modules 520 may be arranged in the second direction, which is the direction of the short side 450, and thus a laser irradiation surface may be formed by the plurality of laser modules.

In FIG. 5, the laser modules 520 are arranged in a single line in the first direction 530 and arranged in a plurality of lines in the second direction, but the present disclosure is not limited thereto. The laser modules 520 may be formed in a plurality of lines in the first direction 530 and formed in a plurality of lines in the second direction 540. Further, while the laser modules 520 are arranged parallel to each other in FIG. 5, the present disclosure is not limited thereto, and the laser modules 520 may be arranged non-parallel to each other.

The handpiece 130 may include at least one each of a first laser module, a second laser module, a third laser module, and a fourth laser module. That is, the handpiece 130 may include at least four laser modules. Further, since each laser module includes at least one laser diode, the handpiece 130 may include at least four laser diodes. The at least four laser diodes may be first to fourth laser diodes.

A laser irradiation surface may be formed on the surface 510 on one side of the handpiece 130, and the surface 510 on one side of the handpiece 130 may be brought into contact with the patient's skin 310 to irradiate the surface 510 on the one side of the handpiece 130 with laser beams so that the treatment effect is achieved over a large area of the patient's skin, and thus the treatment effect may be completed in a short period of time. Therefore, patient and practitioner fatigue may be reduced.

The skin treatment device 100 may guide performing a treatment on the patient by moving the handpiece 130 in the second direction or a direction opposite to the second direction. Further, the handpiece 130 may be provided with markings indicating the direction in which the handpiece should be moved during the treatment. The practitioner may perform the treatment on the patient's skin by moving the handpiece 130 in the second direction or in the direction opposite to the second direction. Therefore, all the laser beams from the first to fourth laser modules may be irradiated to a wide area of the patient's skin. Therefore, the treatment effect on the patient's skin 310 may be achieved by the first to fourth laser modules.

Each laser module may include the same number of laser diodes. Further, the number of first laser modules may be 10 percent or greater and 30 percent or less of the total number of laser modules. The total number of laser modules may be the number of first to fourth laser modules. Further, the number of fourth laser modules may be 25 percent or greater and 40 percent or less of the total number of laser modules. Further, the number of second laser modules may be 15 percent or greater and 25 percent or less of the total number of laser modules. Further, the number of third laser modules may be 25 percent or greater and 35 percent or less of the total number of laser modules.

For example, the laser module array may include five first laser modules, four second laser modules, five third laser modules, and six fourth laser modules. However, the present disclosure is not limited thereto. A ratio of the first laser modules, the second laser modules, the third laser modules, and the fourth laser modules included in the laser module array may be 5:4:5:6. In the case in which this ratio is present, the ratio of the number of laser diodes becomes the same as described above, and it has been experimentally proven that the skin improvement effect is superior to the conventional one.

The laser modules may include different numbers of laser diodes. The handpiece may include the same number of first laser modules, second laser modules, third laser modules, and fourth laser modules. However, the number of first laser diodes included in the first laser module may be 10 percent or greater and 30 percent or less of the total number of laser diodes. Further, the number of fourth laser diodes included in the fourth laser module may be 25 percent or greater and 40 percent or less of the total number of laser diodes. The number of second laser diodes included in the second laser module may be 15 percent or greater and 25 percent or less of the total number of laser diodes. Further, the number of third laser diodes included in the third laser module may be 25 percent or greater and 35 percent or less of the total number of laser diodes.

One or more laser diodes included in a single laser module may be turned on or off simultaneously. However, the present disclosure is not limited thereto, and one or more laser diodes included in a single laser module may be individually turned on or off by the control unit 200.

In the laser module array, the laser modules may be arranged in the following order. As previously described, the second direction 540 may be a direction in which the user moves the handpiece 130. The first laser module, the second laser module, the third laser module, and the fourth laser module may be arranged in order from the second direction toward the direction opposite to the second direction. For example, from the second direction toward the direction opposite to the second direction, a plurality of first laser modules may be arranged, a plurality of second laser modules may be arranged, a plurality of third laser modules may be arranged, and a plurality of fourth laser modules may be arranged. However, the present disclosure is not limited thereto, one first laser module, one second laser module, one third laser module, and one fourth laser module may be arranged from the second direction toward the direction opposite to the second direction to form a small group of laser modules, and a plurality of small groups of laser modules may be arranged from the second direction toward the direction opposite to the second direction to form a laser module array. At least one of the first laser module, the second laser module, and the third laser module may be omitted from one of the plurality of small groups of laser modules. The therapeutic handpiece of the present disclosure may use the laser module array described above to achieve the most optimal treatment effect. This is because the first laser module may remove foreign substances from the surface of the patient's skin 310, the second to fourth laser modules may treat the interior of the patient's skin 310, and the fourth laser module may be positioned relatively opposite to the second direction so that the treatment may be performed on the area of the patient's skin 310 through which the handpiece 130 passes.

As described above, the handpiece 130 may include a plurality of laser modules provided on the surface 510 on one side thereof. Further, at least one laser diode may be included in each laser module. The control unit 200 may adjust a size and shape of a laser irradiation area by turning on or off at least some of the laser diodes included in the plurality of laser modules. Therefore, the skin treatment device 100 may be used not only to provide a treatment over a wide area, but also to provide a treatment over a small area.

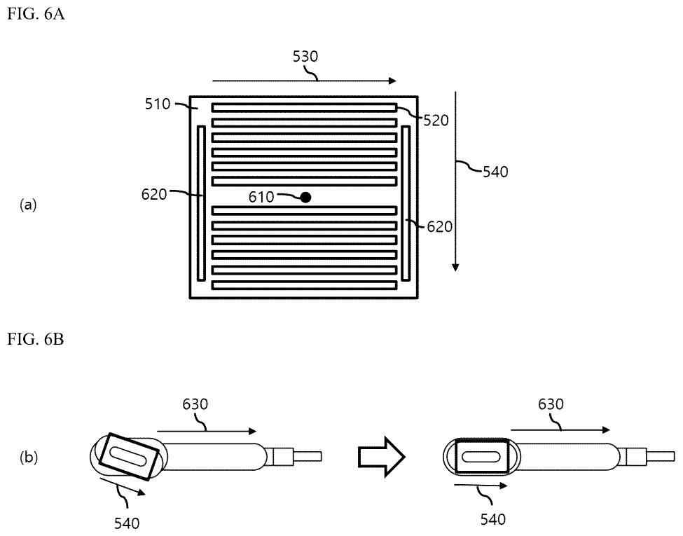

FIGS. 6A and 6B show diagrams for describing a handpiece according to an embodiment of the present disclosure.

According to the present disclosure, a handpiece 130 may include a component for rotating a surface 510 on one side of the handpiece 130. For example, the handpiece 130 may include a rotation shaft 610 perpendicular to the surface 510 on the one side, and the surface 510 on the one side may be rotated relative to the handpiece 130 about the rotation shaft 610. The rotation shaft may be positioned at the center of the surface 510 on the one side.

According to an embodiment of the present disclosure, the control unit 200 may measure a moving direction of the handpiece 130 using at least one of an acceleration sensor and a gyro sensor. Further, the surface 510 on the one side may be rotated around the rotation shaft 610 such that the moving direction of the handpiece 130 matches the second direction 540 or the direction opposite to the second direction. The handpiece 130 may include a driving unit for rotating the surface 510 on the one side around the rotation shaft 610.

Referring to FIG. 6A, according to various embodiments of the present disclosure, the rotation shaft 610 may freely rotate the surface 510 on the one side. The surface 510 on the one side may include a plurality of wheels 620. Rotation shafts of the plurality of wheels 620 may be parallel to the surface 510 on the one side.

Referring to FIG. 6B, the plurality of wheels 620 may assist in rotating the surface 510 on the one side about the rotation shaft 610 relative to the handpiece 130 such that the second direction 540 matches the moving direction of the handpiece 130 when the second direction of the surface 510 on the one side is different from the moving direction 630 of the handpiece 130. This is because when the second direction 540 does not match the moving direction 630 of the handpiece 130, the frictional force between the patient's skin 310 and the plurality of wheels 620 increases, and when the second direction 540 matches the moving direction 630 of the handpiece 130, the frictional force between the patient's skin 310 and the plurality of wheels 620 decreases. Here, the second direction 540 may be a direction fixed to the surface 510 on the one side.

A drive shaft of the wheel may include an encoder. The control unit 200 may measure a distance the handpiece 130 moves on the patient's skin 310 using the encoder. The control unit 200 may control the operation of at least one of the plurality of laser modules on the basis of the movement distance of the handpiece 130. The control unit 200 may turn on or off at least one of the plurality of laser modules according to the movement distance of the handpiece 130. More specifically, the control unit 200 may start turning on at least one of the plurality of laser modules according to the movement distance upon receiving a treatment start signal. Further, the control unit 200 may operate the laser module, and then when the movement distance of the handpiece 130 is greater than or equal to a predetermined threshold movement distance, stop the operation of at least one of the plurality of laser modules. The operation of the skin treatment device 100 may allow the practitioner to perform a treatment only on a predetermined area of the patient's skin 310 without significant effort. Therefore, convenience can be improved for the practitioner.

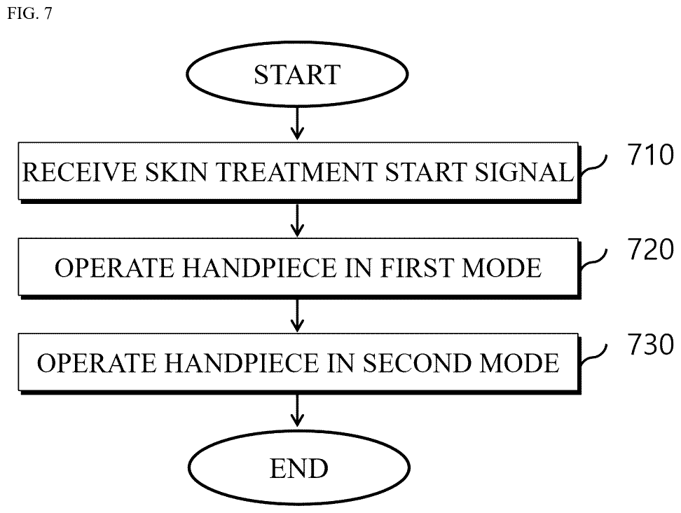

FIG. 7 illustrates an operating method of a skin treatment device according to an embodiment of the present disclosure.

A control unit 200 may perform an operation 710 of receiving a skin treatment start signal. The skin treatment start signal may be acquired by a practitioner or automatically generated when certain conditions are satisfied.

A handpiece 130 may perform an operation 720 of operating the handpiece 130 in a first mode for a predetermined first period of time. The first mode may be a mode in which the intensity of light (laser beams) generated from a first laser module is greater than the intensity of light (laser beams) generated from any one of second to fourth laser modules. For example, the control unit 200 may turn on only the first laser module in the first mode. Alternatively, the control unit 200 may completely turn on the first laser module in the first mode and turn off at least some of the second to fourth laser modules. In this way, by irradiating a patient's skin 310 with laser beams using the first laser module in the first mode, foreign substances on the surface of the patient's skin may be removed. Further, a user may move the handpiece 130 against the patient's skin 310 for the first period of time to remove foreign substances on a surface of a treatment area. The foreign substances may be, for example, the patient's hair. Therefore, after the first mode, the laser beams generated from the second to fourth laser modules may easily penetrate into the patient's skin 310.

The handpiece 130 may perform an operation 730 of operating the handpiece 130 in a second mode after the first period of time. The second mode may be a mode in which the first to fourth laser modules operate according to a predetermined pattern. In the second mode, the operation 730 may be performed for a predetermined second period of time. The second period of time may be relatively longer than the first period of time. The predetermined pattern may include at least one of a radiation time period, a rest time period, a pulse-on time period, a pulse cycle, and the number of pulses during the radiation time period, of at least one of the first to fourth laser modules.

In FIG. 7, although a skin treatment device 100 is described as operating in the first mode and the second mode, the present disclosure is not limited thereto. The skin treatment device 100 may operate in the second mode without the first mode. That is, the skin treatment device 100 may operate the first to fourth laser modules according to the predetermined pattern.

Hereinafter, the predetermined pattern will be described with reference to FIG. 8.

FIG. 8 is a diagram for describing the operation of a skin treatment device according to an embodiment of the present disclosure.

At least one of a radiation time period 810, a rest time period 820, a pulse-on time period 830, and a pulse cycle 840, and the number of pulses during the radiation time period of at least one of first to fourth laser modules that are included in a predetermined pattern may have the following meanings. The predetermined pattern may be different for each of the first to fourth laser modules. For example, the control unit 200 may operate all one or more first laser diodes included in the first laser module according to a predetermined pattern for the first laser module. Further, the control unit 200 may operate all one or more second laser diodes included in the second laser module according to a predetermined pattern for the second laser module. The control unit 200 may operate all one or more third laser diodes included in the third laser module according to a predetermined pattern for the third laser module. The control unit 200 may operate all one or more fourth laser diodes included in the fourth laser module according to a predetermined pattern for the fourth laser module.

However, the present disclosure is not limited thereto, and the control unit 200 may store a predetermined pattern for each laser diode included in the laser module. Therefore, the control unit 200 may control the operation of the handpiece according to the predetermined pattern for each laser diode.

Further, when the handpiece includes a plurality of first laser modules, a plurality of second laser modules, a plurality of third laser modules, and a plurality of fourth laser modules, the control unit 200 may store a predetermined pattern to be used for all of the plurality of first laser modules. In the same manner, the control unit 200 may store a predetermined pattern to be used for all of the plurality of second laser modules and store a predetermined pattern to be used for all of the plurality of third laser modules, and the control unit 200 may store a predetermined pattern to be used for all of the plurality of fourth laser modules. Therefore, laser modules that radiate laser beams of the same wavelength may perform the same operation.

However, the present disclosure is not limited thereto, and the control unit 200 may store different predetermined patterns for each of the plurality of first laser modules. Further, the control unit 200 may have different predetermined patterns for each of the plurality of second laser modules. The control unit 200 may have different predetermined patterns for each of the plurality of third laser modules. The control unit 200 may have different predetermined patterns for each of the plurality of fourth laser modules. Therefore, it is possible to control various patterns.

The radiation time period 810 may be a time period during which at least one of the first to fourth laser modules radiates laser beams by at least one pulse generated by the control unit 200.

The rest time period 820 may be a time period during which at least one pulse is not generated by the control unit 200. During the rest time period 820, at least one of the first to fourth laser modules may not radiate laser beams.

The pulse-on time period 830 may be a time period during which the pulse is high. For example, the laser beams may be irradiated during the pulse-on time period 830. The laser beams may not be irradiated during a pulse-off time period. However, the present disclosure is not limited thereto, and the laser beams may not be irradiated during the pulse-on time period 830, and the laser beams may be irradiated during the pulse-off time period.

The pulse cycle 840 may be the sum of one pulse-on time period and one pulse-off time period.

The number of pulses during the radiation time period 810 may be the number of times pulse transitions from low to high or from high to low during the radiation time period 810. The greater the number of pulses, the more continuously the laser beams may be irradiated.

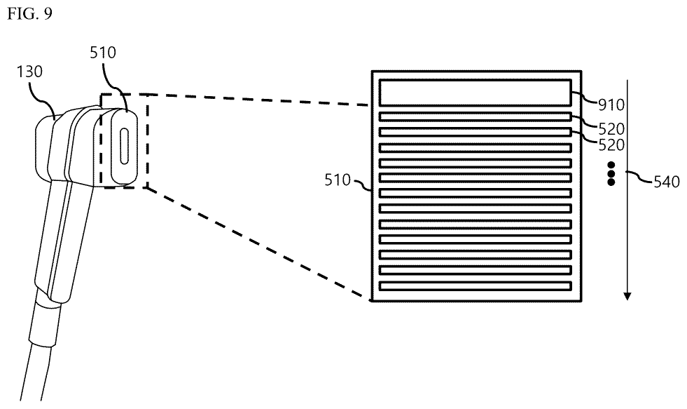

FIG. 9 is a diagram for describing a handpiece according to an embodiment of the present disclosure.

A handpiece 130 may include a skin imaging camera 910 that is positioned to be coplanar with a first laser module, a second laser module, a third laser module, and a fourth laser module and photographs a patient's skin.

The skin imaging camera 910 may be positioned behind a laser module 520 in a second direction 540. That is, when the handpiece 130 is moved in the second direction 540, the laser module 520 may be positioned relatively forward and the skin imaging camera 910 may be positioned behind the laser module 520. Therefore, the skin imaging camera 910 may check the condition of the skin after laser beam radiation. Further, the skin imaging camera 910 may control the turning on or off of the laser module.

The skin imaging camera 910 may capture images of a patient's skin 310, and a skin treatment device 100 may output the images of the patient's skin 310. The images of the patient's skin 310 may be images obtained by magnifying and photographing the patient's skin 310. The patient and the practitioner may check the condition of the patient's skin. Since the images of the patient's skin 310 may be accumulated, the patient and the practitioner may check the process of the patient's skin 310 being improved by the skin treatment device 100.

A control unit 200 may determine at least one of laser setting information and a skin type on the basis of the images of the patient's skin 310. The control unit 200 may apply the images of the patient's skin 310 to a laser setting machine learning model to determine the laser setting information. The laser setting machine learning model may perform machine learning on the basis of the images of the patient's skin 310 treated by a medical professional in the past and setting values of the skin treatment device 100 used by the medical professional. The laser setting machine learning model may be a convolutional neural network (CNN)-based model.

Further, the control unit 200 may apply the images of the patient's skin 310 to a skin type machine learning model to determine the skin type. The skin type machine learning model may perform machine learning on the basis of the images of the patient's skin 310 whose skin type has been classified by the medical professional in the past. The skin type machine learning model may be a CNN-based model.

However, the present disclosure is not limited thereto, and the control unit 200 may determine laser setting information corresponding to the determined skin type. The control unit 200 may include a laser setting information table in which the laser setting information is mapped to the skin type. The control unit 200 may determine laser setting information corresponding to the skin type on the basis of the laser setting information table. The laser setting information may include at least one of a predetermined pattern of laser beams, the intensity of the laser beams, and a treatment time period. Since the predetermined pattern has already been described with reference to FIG. 8, the description thereof will not be repeated.

According to various embodiments of the present disclosure, a memory 230 may store a patient's treatment history and skin type. The control unit 200 may determine laser setting information on the basis of the patient's treatment history and the skin type stored in the memory 230. The control unit 200 may determine whether the patient has received skin treatment within a threshold time period, on the basis of the patient's treatment history. When the patient has received skin treatment within the threshold time period, the control unit 200 may reduce the intensity of the laser beams. This is because lesions may already have formed and be healing inside the patient's skin 310.

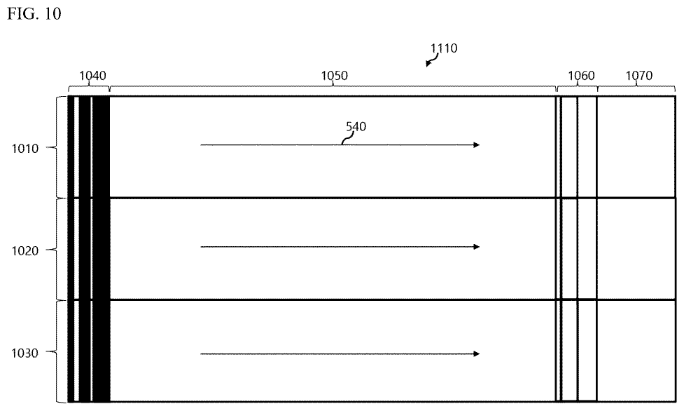

Hereinafter, markers that interact with the skin imaging camera 910 will be described with reference to FIG. 10.

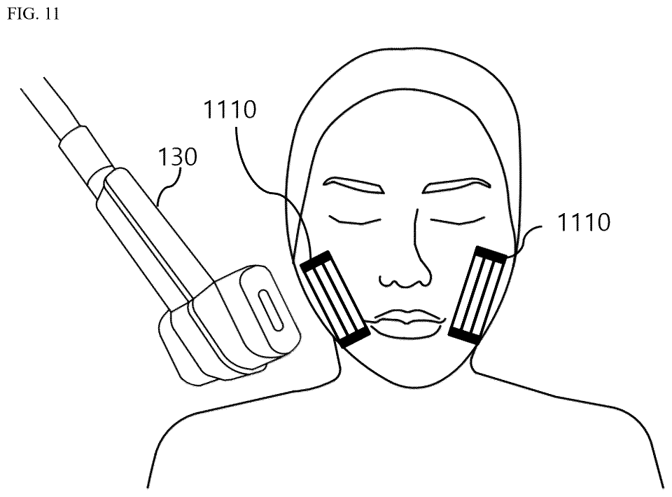

FIG. 10 is a diagram for describing markers according to an embodiment of the present disclosure. FIG. 11 is a diagram illustrating an example of performing a laser treatment using markers according to an embodiment of the present disclosure.

Referring to FIG. 11, a marker 1110 may be a sticker temporarily attached to a patient's skin. Markers may be attached to the patient's skin 310 prior to a treatment. Since the marker 1110 clearly identifies a treatment area, the practitioner may easily identify the treatment area, and thus medical accidents may be prevented and convenience may be increased for the practitioner. Further, since the patient may also identify the treatment area, misunderstandings with the practitioner may be prevented. Further, the marker 1110 may be made of a material that transmits the wavelength of laser beams generated from the first to fourth laser modules.

The marker 1110 may include a start point marker 1040, a treatment section 1050, an end point marker 1060, and an additional section 1070. The marker 1110 may include a plurality of rows 1010, 1020, and 1030.

Referring to FIG. 10 together with FIG. 9, the skin imaging camera 910 may capture an image of the start point marker 1040 pre-marked on the surface of the patient's skin 310. The control unit 200 may recognize the start point marker in the image captured by the skin imaging camera 910. The control unit 200 may recognize the start point marker by matching the captured image with the predetermined pattern. The start point marker 1040 may have a specific pattern. For example, the specific pattern may be a quick response (QR) code or barcode. The skin imaging camera 910 may be a QR code reader or a barcode reader.

When the skin imaging camera 910 captures an image of the start point marker 1040 pre-marked on the surface of the patient's skin 310, the control unit 200 may control any one of the first to fourth laser modules to be turned on. Controlling any one of the first to fourth laser modules to turn on may mean performing a laser treatment.

The handpiece 130 may treat the patient's skin 310 by radiating the treatment section 1050 with laser beams. The treatment section 1050 may not be marked. The handpiece 130 may perform the treatment while moving in the second direction 540.

When the skin imaging camera 910 captures an image of the end point marker 1060 pre-marked on the surface of the patient's skin, the control unit 200 may control any one of the first to fourth laser modules to be turned off. As previously described, since the skin imaging camera 910 moves behind the laser module, the marker 1110 may include the additional section 1070. When the skin imaging camera 910 is positioned at the end point marker 1060, the laser module may be positioned at the additional section 1070. That is, sections in which the laser module radiates laser beams may be the treatment section 1050, the end point marker 1060, and the additional section 1070.

The end point marker 1060 may be a thin marker and thus may not interfere with the laser treatment. Further, the end point marker 1060 may be made of a material capable of transmitting the laser beams irradiated from the first to fourth laser modules.

The marker 1110 may include the plurality of rows 1010, 1020, and 1030. The practitioner may perform the treatment on a first row 1010 and then perform the treatment on the second row 1020 and third row 1030. Although three rows are illustrated in FIG. 10, the present disclosure is not limited thereto. The number of the plurality of rows 1010, 1020, and 1030 may be smaller or greater than three.

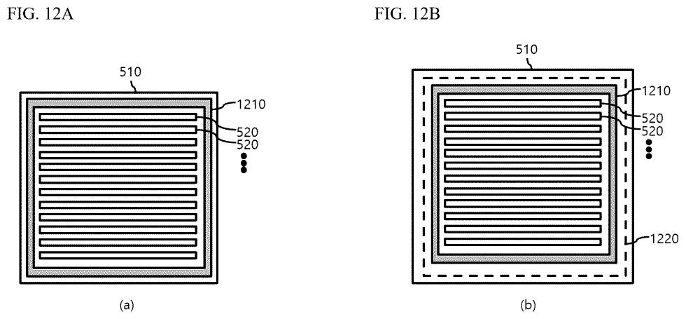

FIGS. 12A and 12B show diagrams for describing a handpiece according to an embodiment of the present disclosure.

Referring to FIG. 12A, the skin treatment device 100 may include a cooling unit 1210 for controlling a temperature of the patient's skin 310 during a laser treatment. The cooling unit 1210 may surround the laser modules 520. The cooling unit 1210 may maintain the temperature of the patient's skin 310 at a temperature less than or equal to a certain temperature to prevent burns on the surface of the patient's skin 310. The cooling unit 1210 may be implemented using various cooling methods, such as cryogenic spray or a thermoelectric cooling system.

The handpiece 130 may include a temperature sensor for measuring the temperature of the patient's skin. The control unit 200 may control the laser modules 520 to maintain a rest time period longer when the temperature of the patient's skin 310 measured by the temperature sensor is greater than or equal to a threshold temperature. Therefore, the skin treatment device 100 may control the temperature of the patient's skin 310 to be maintained at a temperature less than or equal to the threshold temperature.

Referring to FIG. 12B, the skin treatment device 100 may include an elasticity measurement unit for measuring skin elasticity in at least a portion of a region 1220 indicated by a dotted line. The elasticity measurement unit may be at least one transducer formed on the surface 510 on one side of the handpiece 130. The elasticity measurement unit may radiate predetermined ultrasound waves and receive ultrasound waves reflected by the patient's skin 310. The elasticity measurement unit may radiate ultrasound wave waves having at least one frequency and measure the frequency of ultrasound waves reflected by the patient's skin 310. The control unit 200 may analyze the frequency of the reflected ultrasound waves to measure the elasticity of the patient's skin 310. More specifically, the control unit 200 may measure the signal intensity of the reflected ultrasound waves at each frequency, acquire at least one peak frequency with the strongest intensity, and acquire the elasticity of the patient's skin corresponding to the corresponding frequency. The control unit 200 may store an elasticity acquisition table in which the elasticity of the patient's skin is mapped to at least one peak frequency of the reflected ultrasound waves. The control unit 200 may acquire the elasticity of the patient's skin corresponding to at least one peak frequency using the elasticity acquisition table. The control unit 200 may acquire the elasticity of the patient's skin using a skin elasticity machine learning model. The skin elasticity machine learning model may be a machine-learned model using training data that labels the elasticity of the patient's skin according to the intensity of the signal at each frequency of the reflected ultrasound waves. The control unit 200 may acquire the elasticity of the current patient's skin by applying the intensity of the signal of the reflected ultrasound waves on the current patient's skin at each frequency to the skin elasticity machine learning model. Based on the elasticity measurement unit, the skin treatment device 100 may measure skin elasticity before and after the treatment. Further, the elasticity measurement unit may determine the thickness of the patient's skin in a corresponding area on the basis of ultrasound waves of a specific wavelength.

The practitioner may evaluate the effectiveness of the treatment on the basis of the measured data, and accordingly, the measured data may be used to adjust the treatment method. For example, the control unit 200 may adjust a pulse duration time period of the laser radiation according to the condition of the patient's skin 310. When the patient's skin 310 is sensitive or thin, the control unit 200 may reduce at least one of the radiation time period 810 and the pulse-on time period 830 to prevent skin damage. Further, when the patient's skin 310 is thick or elastic, the control unit 200 may increase at least one of the radiation time period 810 and the pulse-on time period 830 to acquire a result of the treatment. The control unit 200 increases at least one of the radiation time period 810 and the pulse-on time period 830 to allow the laser energy to penetrate deeper into the skin when the skin is thick. Further, the control unit 200 may control the fourth laser module to radiate more strongly than the second laser module when the skin is thicker, and control the second laser module to radiate more strongly than the fourth laser module when the skin is thinner. This is because, as previously described, the laser beams irradiated from the fourth laser module may penetrate into the skin deeply.

Further, the skin treatment device 100 may clearly inform the patient of the skin improvement effects by displaying the measured data numerically. Further, the preference for the device may increase according to the treatment effect by the skin treatment device 100.

Further, the control unit 200 may adjust at least one of the radiation time period 810 and the pulse-on time period 830 to be longer in an early part of a total treatment time period, and adjust at least one of the radiation time period 810 and the pulse-on time period 830 to be shorter in a later part of the total treatment time period. This is because the patient's skin will be damaged during the treatment, and therefore the laser beams are used to perform only a fine treatment on the latter part to prevent excessive damage to the patient's skin.

Further, the control unit 200 may be programmed to adjust the pulse duration time period according to the patient's pain tolerance. For patients with low pain tolerance, at least one of the radiation time period 810 and the pulse-on time period 830 may be reduced to minimize discomfort during treatment. The patient's pain tolerance may be determined by the practitioner asking the patient during the treatment.

The elasticity measurement unit may be used as a component for vibrating the skin during skin treatment. The elasticity measurement unit may provide massage or vibration to the skin during skin treatment. The massage or vibration of the elasticity measurement unit may increase blood circulation and lymphatic drainage in the skin to improve the effectiveness of skin treatment.

Referring to FIG. 12B, the skin treatment device 100 may include a medication application unit in at least a portion of the region 1220 indicated by the dotted line. The medication application unit may be at least one fine hole formed on the surface 510 on one side of the handpiece 130. The handpiece 130 may use the medication application unit to spray the medication inside the handpiece 130 to the outside. The handpiece 130 may include a pressure generation unit that generates high pressure and a medication storage unit. The handpiece 130 may spray a predetermined amount of medication of the medication storage unit from the medication application unit onto the patient's skin 310 on the basis of the high pressure of the pressure generation unit. The medication may promote skin regeneration and relieve pain. Therefore, the practitioner may avoid the hassle of applying medication separately, and since the medication application and the treatment are performed almost simultaneously, a treatment time period may be reduced. Further, not only may the patient experience little to no pain from the treatment, but convenience may also increase as the treatment time period is reduced.

While the present disclosure has been particularly described with reference to various embodiments, it will be understood by those skilled in the art that various changes in form and details may be made without departing from the spirit and scope of the present disclosure. Therefore, the disclosed embodiments should be considered from an exemplary point of view for description rather than a limiting point of view. The scope of the present disclosure is indicated in the claims rather than the foregoing description, and all differences within the scope equivalent thereto should be construed as being included in the present disclosure.

Meanwhile, the above-described embodiments of the present disclosure can be written as a program that can be executed on a computer and can be implemented in a general purpose digital computer that operates a program using computer-readable recording media. The computer-readable recording media can include storage media such as magnetic storage media (e.g., a ROM, a floppy disk, a hard disk, etc.) and optically readable media (e.g., a compact disc read only memory (CD-ROM), a digital video disc (DVD), etc.).

What is claimed is:

1. A medical skin treatment device comprising:
a control unit configured to control operation of the skin treatment device;
a main body which includes a transfer unit and a power supply; and
a handpiece which receives power from the power supply of the main body and is controlled by the control unit and of which one side is brought into contact with a patient's skin to irradiate the skin with laser beams,
wherein the handpiece includes at least four different laser diodes,
the at least four different laser diodes radiate laser beams of four different wavelengths and include first laser diodes, second laser diodes, third laser diodes, and fourth laser diodes,
the number of the first laser diodes is 30 percent or less of the total number of the laser diodes, and
the number of the fourth laser diodes is 25 percent or greater of the total number of the laser diodes.

2. The medical skin treatment device of claim 1, wherein the first laser diodes are laser diodes for burning hair on a surface of the patient's skin,
the second to fourth laser diodes are laser diodes for penetrating the patient's skin to form a lesion under the patient's skin,
a depth at which the second laser diode penetrates into the skin is smaller than a depth at which the third laser diode penetrates into the skin, and
the depth at which the third laser diode penetrates into the skin is smaller than a depth at which the fourth laser diode penetrates into the skin.

3. The medical skin treatment device of claim 2, wherein a wavelength of light generated from the first laser diode is shortest,
a wavelength of light generated from the second laser diode is longer than the wavelength of the light generated from the first laser diode,
a wavelength of light generated from the third laser diode is longer than the wavelength of the light generated from the second laser diode, and
a wavelength of light generated from the fourth laser diode is longer than the wavelength of the light generated from the third laser diode.

4. The medical skin treatment device of claim 3, wherein the wavelength of the light generated from the first laser diode is 750 nanometers or more and 770 nanometers or less,
the wavelength of the light generated from the second laser diode is 800 nanometers or more and 820 nanometers or less,
the wavelength of the light generated from the third laser diode is 930 nanometers or more and 950 nanometers or less, and
the wavelength of the light generated from the fourth laser diode is 1,050 nanometers or more and 1,070 nanometers or less.

5. The medical skin treatment device of claim 4, wherein:
the first laser diodes are included in a first laser module;
the second laser diodes are included in a second laser module;
the third laser diodes are included in a third laser module;
the fourth laser diodes are included in a fourth laser module; and
the first to fourth laser modules are arranged to be coplanar with the handpiece.

6. The medical skin treatment device of claim 5, wherein the first laser module, the second laser module, the third laser module, and the fourth laser module have a form of laser diodes arranged on a rectangular substrate, and
wherein the rectangular substrate has long sides in a first direction and short sides in a second direction perpendicular to the first direction,
the first laser module, the second laser module, the third laser module, and the fourth laser module are arranged in parallel in the second direction to form a laser module array, and
the laser module array includes five first laser modules, four second laser modules, five third laser modules, and six fourth laser modules.

7. The medical skin treatment device of claim 5, wherein the control unit is configured to:
control an intensity of light generated from the first laser module to be greater than an intensity of light generated from any one of the second to fourth laser modules for a predetermined first period of time; and
operate the first to fourth laser modules according to a predetermined pattern after the first period of time.

8. The medical skin treatment device of claim 6, wherein the handpiece includes a skin imaging camera that is positioned to be coplanar with the first laser module, the second laser module, the third laser module, and the fourth laser module and photographs the patient's skin,
when the skin imaging camera photographs a start point marker pre-marked on the surface of the patient's skin, the control unit controls any one of the first to fourth laser modules to be turned on, and
when the skin imaging camera photographs an end point marker pre-marked on the surface of the patient's skin, the control unit controls any one of the first to fourth laser modules to be turned off.
